(19) United States
(12) Patent Application Publication
(10) Pub. No.: US 2007/0049873 A1
(43) Pub. Date: Mar. 1, 2007

(54) Impulse chamber for jet delivery device

(75) Inventors: Torben Strom Hansen, Copenhagen (DK); Claus Schmidt Moller, Fredensborg (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/493,095

(22) Filed: Jul. 26, 2006

Related U.S. Application Data
(63) Continuation of application No. PCT/DK05/00042, filed on Jan. 24, 2005.

(30) Foreign Application Priority Data
Jan. 26, 2004 (DK) PA 2004 00099
Feb. 12, 2004 (DK) PA 2004 00215

(51) Int. Cl. A61M 5/00

(52) U.S. Cl. 604/187

(57) ABSTRACT

The invention relates to an impulse chamber which can be used for expelling an amount of a fluid compound at a high pressure. The impulse chamber comprises a variable-volume impulse chamber adapted for containing a volume of a flowable drug, an outlet nozzle in fluid communication with the impulse chamber and being adapted to be arranged against the skin of a subject, and a fluid inlet in fluid communication with the impulse chamber. The impulse chamber is defined substantially by a deformable chamber portion, such that deformation thereof reduces the volume of the cavity. In an exemplary embodiment the compressible chamber portion is in the form of an elastomeric tube, this providing a simple, yet reliable and cost-effective impulse chamber unit.

### BACKGROUND OF THE INVENTION

Subcutaneous and intramuscular delivery of liquid drugs by injection is common in the medical arts. As some medications, such as insulin, must be given frequently by injection to an individual, it is desirable that the injections can be performed easily.

Many patients dislike needle injections due to pain or fear for needles. Further, blood-borne pathogens, such as HIV and hepatitis, can be transmitted to health care workers by accidental needle-sticks. Also, the disposal of used needles is a growing concern. This disposal presents a problem to individuals other than healthcare workers. Children, for example, may find used needles in the trash, putting them at risk of contracting infection. Discarded needles likewise pose a risk to waste disposal workers.

In efforts to minimize the fears and risks associated with needle injections, several types of needle-free jet injectors have been developed. These devices make the drug penetrate the skin using a high velocity fluid jet, and thus deliver medication into the tissue of a patient. In order to accomplish this, a force is exerted on the liquid medication. Jet injectors, in general, contain a fluid drug which has been transferred into a chamber having a small orifice at one end. A drive element, e.g. a ram, is accelerated using either a coil spring or a compressed gas energy source. The ram impacts a plunger, which in turn creates a high pressure impulse within the chamber. This pressure impulse ejects the fluid medicament through the orifice at high velocity, piercing the skin. The energy source continues to apply a force to the plunger, which quickly propels the drug through the opening in the skin, emptying the syringe in a fraction of a second.

Most jet injectors comprise a chamber, an outlet at a first end of the chamber, and a piston at an opposed second end, the outlet being a jet nozzle. The piston is typically driven by a drive mechanism such as a compressed spring or an expandable gas, or other means that can be released to deliver a force to expel the liquid under a high pressure from the chamber.

Such jet injectors are typically large as compared to needle injectors, e.g. pen type needle injectors, which of course is a disadvantage in relation to handling and transportation. Further the size and shape of such apparatuses can have an intimidating effect on many patients.

The drive mechanism of one-stage jet injectors typically influences the piston with a linearly decreasing force, i.e. the liquid is expelled under a steadily decreasing pressure, over the duration of the injection. Thus, when the derma has been penetrated and the jet enters the fragile subcutaneous tissue, the pressure of the liquid jet is still high, possibly causing lesions to the subcutaneous tissue, e.g. damaging tissue cells, nerve fibres and fine blood vessels. This may cause haemorrhage and pain and trauma for the patient. Further the damage to the tissue can trigger an immune reaction in the tissue, causing the chemical environment at the injection site to change. This can influence the effect of the injected substance, which of course is highly undesirable. If the pressure of the jet is too high, the jet passes through the subcutaneous layer into connecting tissue or muscular tissue underneath. For example, in case the injected liquid is insulin, it is essential that the insulin is not delivered to the connecting tissue. This tissue has a lot of blood vessels and will absorb the insulin too quickly, with the risk of resulting in insulin chock.

To provide a better control over the jet injection, the drive means may be adapted to provide a two-stage injection, i.e. a first penetrating burst of drug at a high pressure followed by a subsequent delivery of the remaining amount of drug at a lower pressure. More specifically, the derma is first penetrated by a short, intense jet under high pressure where after the main part of the medical compound is injected under a much lower pressure. By utilizing this principle the overall energy that has to be absorbed by the skin tissue is substantially decreased, and consequently the damage to the tissue is lowered.

Several jet injection devices are known to utilize this principle, for example as disclosed in EP 879 609, EP 1 161961, WO 01/47586 and WO 02/49697, hereby incorporated by reference. These documents disclose jet injection devices wherein a dose of a medical compound is expelled from a chamber and where the drive mechanism acting on the chamber is adapted to deliver two bursts. The driving mechanisms of these devices all have rather complicated structures because they need to be able to deliver two distinct bursts of force.

A different approach is known from WO 03/000320 disclosing a jet injection device comprising a disposable jet injection unit having an impulse chamber with an outlet nozzle. The impulse chamber has generally rigid walls, however, a section comprises a resilient wall portion. The jet injection unit further has means for connecting the impulse chamber in fluid communication with a reservoir for a liquid medical compound, and thrust beams for deforming the resilient wall portion, the thrust beams being moved by a drive mechanism in the form of an over-the-centre leaf spring. WO 2004/039438 discloses a similar jet injection device comprising a bi-stable spring for actuation of the impulse chamber.

When the outlet nozzle is arranged against the skin of a subject and the spring forcing the thrust beams against the flexible wall portion of the chamber is released, the resilient wall portion is deformed, whereby a volume of a liquid contained in the impulse chamber is injected from the chamber under a high pressure, creating a liquid jet for penetrating the skin and establishing a channel therethrough. Thereupon a dose from the medical compound reservoir can be injected into the body through the impulse chamber and the established channel. The jet injection unit is adapted to be discarded upon use. A similar technology is disclosed in WO 01/30419 wherein the drive mechanism comprises a bi-axially curved spring.

Whenever a medical device comes in contact with e.g. the skin of a patient or is handled there is a risk of contamination of the device. In the case of injection devices, the jet nozzle typically comes in contact with the skin. Therefore in the above mentioned prior art devices the entire jet injection unit comprising jet nozzle, fluid chamber, fluid connection and drive mechanism is supposed to be disposed of after use.

In view of the above, it is an object of the present invention to provide an impulse chamber unit which can be used in combination with a jet injection device, the impulse chamber unit being simple and compact in design, thus allowing for cost-efficient manufacture, e.g. as a single-use disposable unit.

It is a further object to provide an impulse imparting mechanism to be used in combination with the impulse chamber of the invention.

It is a further object to provide an impulse chamber unit which can be used in combination with a jet injection device adapted to receive a conventional cartridge containing a liquid drug, the impulse chamber unit being simple and compact in design, thus allowing for cost-efficient manufacture, e.g. as a single-use disposable unit.

It is a yet further object of the invention to provide an impulse chamber and reservoir configuration which allows a compact and handy jet injection device to be provided.

It is a further object to provide a jet injection device that can be modeled similar in function and form with a conventional pen type injector, to make the patient comfortable with the jet injection device, and so that the jet injection device can easily be utilized by a non-professional user, e.g. a insulin requiring diabetic.

### DISCLOSURE OF THE INVENTION

In the disclosure of the present invention, embodiments and aspects will be described which will address one or more of the above objects or which will address objects apparent from the below disclosure as well as from the description of exemplary embodiments.

Correspondingly, in a first aspect an impulse chamber unit is provided comprising a deformable chamber portion, the outer surface of the chamber portion being substantially free, a variable-volume impulse chamber defined at least partially by the deformable chamber portion and adapted for containing a volume of a flowable drug, an outlet nozzle in fluid communication with the impulse chamber and being adapted to be arranged against a skin surface of a subject, and a fluid inlet for the impulse chamber, wherein deformation of the chamber portion reduces the volume of the impulse chamber. In a preferred embodiment the variable-volume impulse chamber is defined substantially by the deformable chamber portion.

The chamber portion may be in the form of a deformable polymeric tube comprising proximal and distal end portions. The polymeric tube may be formed from any suitable material allowing the desired deformation thereof and the corresponding volume reduction. For example, the tube may be formed from a thermoplastic elastomeric material (TPE). Although these materials are elastic to a certain degree, a tube formed from such a material may be plastically deformed during deformation, however, for a disposable impulse chamber unit this is acceptable. The tube may have any desirable cross-sectional configuration, e.g. circular.

Alternatively the tube may be formed from an elastomeric material such as silicone rubber. Due to its elastic properties only a small amount of energy is absorbed when a tube made from such a material is deformed. Also, such a material will allow the chamber to substantially regain its configuration when the deforming force seizes.

As elastomeric materials such as silicone rubber typically are almost incompressible, deformation of the tube under well-defined, enveloped conditions can with simple measures reliably provide high precision skin penetrating jets. Further, a rubber tube is simple and cost-efficient to manufacture and takes up little space during transportation. Also, a rubber tube is robust with relation to impacts occurring during use and transportation.

By varying the dimensions and the configuration of the impulse chamber it is possible to vary the characteristics of the jet under unchanged conditions for the surrounding structures. For example, for a given outer tube diameter the inner diameter may be varied, this resulting in jet injections of different volumes without having to necessarily modify the impact or mounting means.

Although the term “deformable” literally applies to all materials to a certain agree, it will be apparent to the skilled person that within the context of the present invention, the term deformable relates to the impact of forces on an impulse chamber which are relevant in the technical field of jet injection.

The tubular member may be arranged between proximal and distal closure portions, the proximal closure portion comprising the fluid inlet in fluid communication with the impulse chamber, and the distal closure portion comprising an outlet nozzle in fluid communication with the impulse chamber. The closure portions may be in the form of separate closure members, which members may be of unitary construction or may comprise a number of separate members. Alternatively, the closure portions may be formed integrally with the tubular member. For example, when the polymeric tube is formed from a material such as TPE the outlet nozzle may be formed integrally with the tube, e.g. as a unitary injection moulded unit. In case the polymeric tube is formed from a rubber-like material the fluid inlet may be provided by a portion of the elastomeric tube being penetratable by a pointed needle, i.e. the tube having a closed proximal end. The closure members may be provided with additional functions. For example, the fluid inlet may be in the form of a conduit member, e.g. a pointed hollow needle, projecting from the proximal closure portion. In an alternative embodiment only a single distal closure member comprising an outlet nozzle is provided, the proximal open end of the tube providing the fluid inlet. When separate members are attached to one or both ends of the elastomeric tube, the outer surface of the tube may be left substantially free, this providing a very compact unit.

Although reference is made to a single nozzle (or aperture), the nozzle of the invention may comprise any desired number of additional apertures. Further, the nozzle may comprise a pointed hollow needle adapted to penetrate a superficial layer of the skin of a user, thereby aiding the jet of drug to create an opening in the skin from the surface to the subcutaneous space. Such a needle may be relatively short, e.g. 1 mm or less.

In an exemplary embodiment the impulse chamber unit of the invention is provided in combination with a mounting device comprising a mounting cavity configured to receive the deformable chamber portion, the mounting unit being adapted to replaceably receive the impulse chamber unit in locking engagement. The mounting cavity may be adapted to receive the chamber portion in a substantially form-fitting relationship. By arranging the deformable chamber portion in a substantially form-fitting structure, the internal volume of the impulse chamber will be reduced in an effective and predictable way during deformation. Advantageously, the deformable chamber portion has a tubular configuration with a substantially constant outer diameter along the length thereof, the cavity being in the form of a bore having a diameter substantially the same as the deformable chamber portion, the bore comprising an opening allowing an impulse generating means to engage a portion of the deformable chamber portion. Alternatively the cavity will only partially engage the outer surface of the deformable chamber portion, this allowing the chamber to more freely deform, e.g. to be flattened. Indeed, as long as the impulse chamber unit is arranged in the mounting cavity the outer surface of the chamber portion is no longer free, however, when the impulse chamber is removed from the mounting cavity the surface is again free. Thus, the term “free” is to be interpreted corresponding to this situation of use.

The impulse chamber unit and the mounting device may comprise releasable mating coupling means allowing the impulse chamber unit to be secured to the mounting device in a situation of use, e.g. in the form of a bayonet or a threaded coupling, a frictional fit, or by a releasable locking means.

The mounting device may comprise connection means for arranging the fluid inlet in fluid communication with an interior of a drug reservoir, as well as impulse generating means for displacing a portion of the deformable chamber portion, thereby reducing the volume of the impulse chamber and thereby expelling an amount of a liquid drug contained in the impulse chamber through the outlet nozzle, the impulse generating means being adapted to create a pressure within the impulse chamber for injecting the liquid drug through the outlet nozzle and into the subject through the skin. The impulse generating means may be of any suitable configuration, but will typically comprise an impact member adapted to engage the resilient chamber portion. The drive energy may be provided by any suitable means allowing a rapid release of energy to the impulse chamber, e.g. mechanical compression or torsion springs, compressed gas, pneumatic or electromechanical actuators. In an alternative embodiment the (first) mounting device is adapted to be arranged in a second mounting device comprising the impulse generating means.

The mounting device may be adapted to cooperate with e.g. a conventional-type drug delivery device such as a manually operated injection device of the pen type, or with an electronically controlled motorized injection device, e.g. the mounting device may be adapted to be mounted as a “pre-unit” in place of a conventional hypodermic needle. In this way a two-stage device is provided, the impulse chamber serving as a first-stage impulse generating means and subsequently as a flow conduit for the second-stage drug injection provided by actuation of the drug delivery device. Advantageously, release of the impulse generating means is coupled to the actuation of the drug delivery means of the drug delivery device.

In an exemplary embodiment, the mounting device and the impulse generating means are provided in the form of a two-stage jet injection device adapted to receive or comprising a reservoir with a liquid drug, and further comprising drive means for expelling an amount of drug from the reservoir at a reduced pressure relative to the impulse generating means. Preferably the device comprises means for selectable setting a dose of drug to be expelled, means for actuating the impulse generating means and the drive means, and actuatable release means, whereby actuation of the release means first causes release of the impulse generating means thereby expelling an amount of drug from the impulse chamber through the outlet nozzle, followed by release of the drive means for subsequent expelling of the set dose from the reservoir via the impulse chamber through the outlet nozzle.

In an exemplary embodiment actuation of the dose setting means also serves to initialize the impulse generating means, e.g. straining a spring member.

Advantageously, the impulse generating means comprises a piston adapted for deforming the chamber portion, a rotatable cam for moving the piston and a drive mechanism for rotating the cam. In a preferred embodiment the cam is rotated by means of a torsion spring, this allowing the user to directly strain the spring by a rotational action. This type of actuation is advantageous as it resembles the dose setting means used in most injection devices of the pen type, thus also allowing the spring actuation and the dose setting to be coupled to each other in a simple and reliable manner.

Preferably, each impulse chamber unit will be supplied to the user in a sealed, sterile enclosure, e.g. corresponding to a sterile hypodermic needle.

As used herein, the term “drug” is meant to encompass any drug-containing flowable medicine or medicament capable of being passed through a nozzle under high pressure in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with references to the drawings, wherein

FIG. 1 shows a first embodiment of an impulse chamber unit,

FIG. 2A shows a second embodiment of an impulse chamber unit,

FIG. 2B shows a cross-sectional view of a further embodiment of an impulse chamber unit mounted in a mounting unit,

FIG. 3 shows a cross-sectional schematic representation of a two-stage jet injection device,

FIGS. 4A and 4B show cross-sectional schematic representations of an impulse chamber unit mounted in an impulse generating unit,

FIG. 5 shows a schematic representation of an impulse generating mechanism,

FIG. 6 shows in a partial cut-away representation an embodiment of a two-stage jet injection device, and

FIGS. 7A and 7B show diagrams of the pressure-time relationship for two-stage jet injection.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

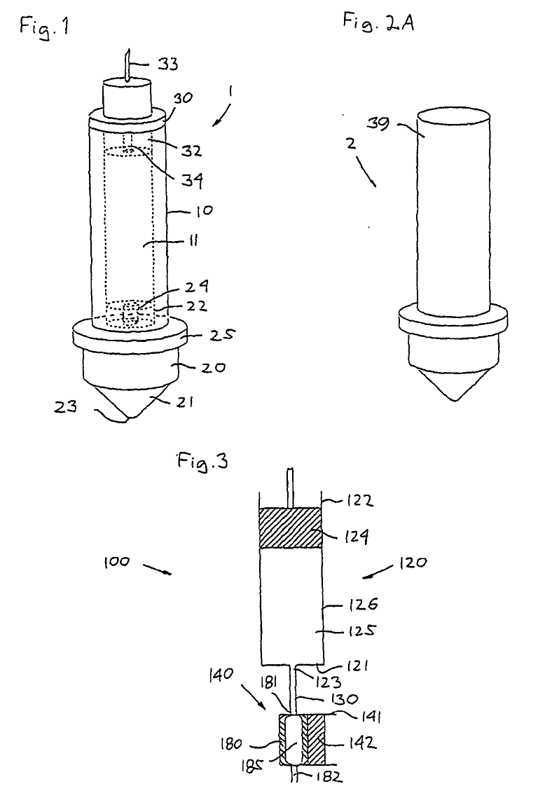

FIG. 1 shows a perspective view of an impulse chamber unit 1 comprising a deformable chamber portion in the form of an elastomeric tube member 10, a distal nozzle portion closure member 20 and a proximal fluid inlet closure member 30. The unit comprises a tubular variable-volume impulse chamber 11 adapted for containing a volume of a flowable drug, the impulse chamber being defined substantially by the tube member, the two closure members merely providing the end portions of the impulse chamber. Alternatively the closure members may comprise extensions making up a portion of the impulse chamber. The distal member comprises a distal conical portion 21, a proximal mounting portion 22 for the tube, a distal-most jet nozzle 23 terminating at the tip portion of the conical portion, an internal bore 24 providing fluid communication between the jet nozzle and the interior of the impulse chamber, and a circumferential flange portion 25. The proximal member comprises a proximal-most pointed hollow needle 33 adapted to sealingly penetrate an elastomeric septum of a drug supply, a distal mounting portion 32 for the tube, and an internal bore 34 providing fluid communication between the needle and the interior of the impulse chamber. Depending on the material forming the tube, either of the closure members may be formed integrally with the tube, e.g. by injection molding.

The volume of the impulse chamber may be in the order of 5-20 μl, however, other volumes may be relevant. The closure members, when formed separately, are advantageously manufactured by injection moulding using a suitable medical-grade polymeric material.

When the elastomeric tube in a situation of use is deformed by impact action, a fluid contained in the impulse chamber is pressurized and expelled through the jet nozzle outlet opening at a high pressure. As appears, in the embodiment of FIG. 1 the impulse chamber may be in permanent fluid communication with a fluid supply, e.g. drug reservoir, which means that the drug is forced not only out through the nozzle but is also forced rearwards through the inlet means back in the reservoir. Indeed, this is not desirable for which reason the flow resistance of the inlet and outlet openings should be chosen such that only an acceptable small volume of drug (if any) is transferred back to the reservoir. Dependent e.g. upon the pressure generated in the impulse chamber by the impulse generating means, the duration of the injection, the viscosity of the liquid drug and the configuration of the nozzle and the fluid inlet, the flow resistance in the nozzle and the fluid inlet should be chosen in accordance with the desired properties for a given jet injection assembly. For example, the flow resistance in the nozzle and the fluid inlet may be chosen to allow a backflow of e.g. less than 1%, less than 5%, less than 10% or less than 15% for a given configuration of a jet injection assembly. To even more effectively protect the cartridge from pressure waves and/or to ideally prevent any backflow, the fluid inlet means may be provided with check valve means, e.g. a lip or ball valve. Further, deformation of the impulse chamber will send shockwaves in the proximal direction towards a fluid supply. If the fluid supply is a reservoir in the form of a traditional glass cartridge, there is a risk of damaging the cartridge. Correspondingly, with an appropriate dimensioning of the fluid inlet the major part of the shockwave may be prevented from reaching such a glass cartridge.

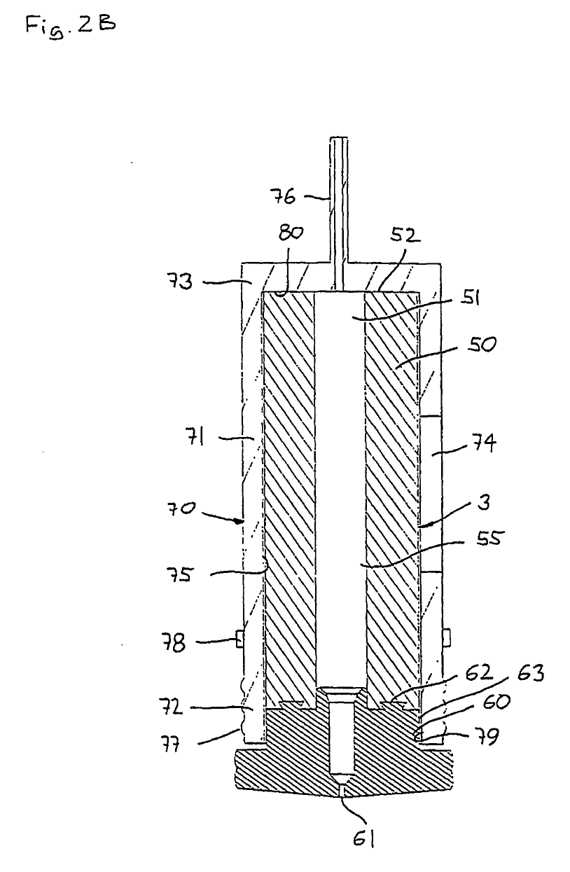

FIG. 2A shows an alternative embodiment 2 in which a polymeric tube is integrally formed with a closed proximal end 39, the proximal end forming a self-sealing needle penetratable septum serving as an inlet means. Indeed, also the above-described proximal member 30 may be provided with a corresponding septum instead of the needle. FIG. 2B shows a further alternative embodiment in which an impulse chamber unit 3 is mounted in a mounting unit (or mounting device) 70.

More specifically, the impulse chamber unit comprises a deformable elastomeric tube member 50 and a distal nozzle closure member 60 in combination defining an impulse chamber 55, the nozzle member comprising a jet nozzle 61. In contrast to the above-described embodiments the impulse chamber unit comprises a generally open proximal end portion with the fluid inlet being defined by the open proximal end 51 of the elastomeric tube. In the shown embodiment the impulse chamber is manufactured by two-component injection moulding, e.g. first the nozzle portion is moulded from a suitable polymer (e.g. polycarbonate) where after the tube is injection moulded onto the nozzle portion using a suitable thermoplastic elastomeric material. To provide a secure connection between the two components the proximal surface of the nozzle portion is advantageously provided with a circumferential ridge structure 62 as shown.

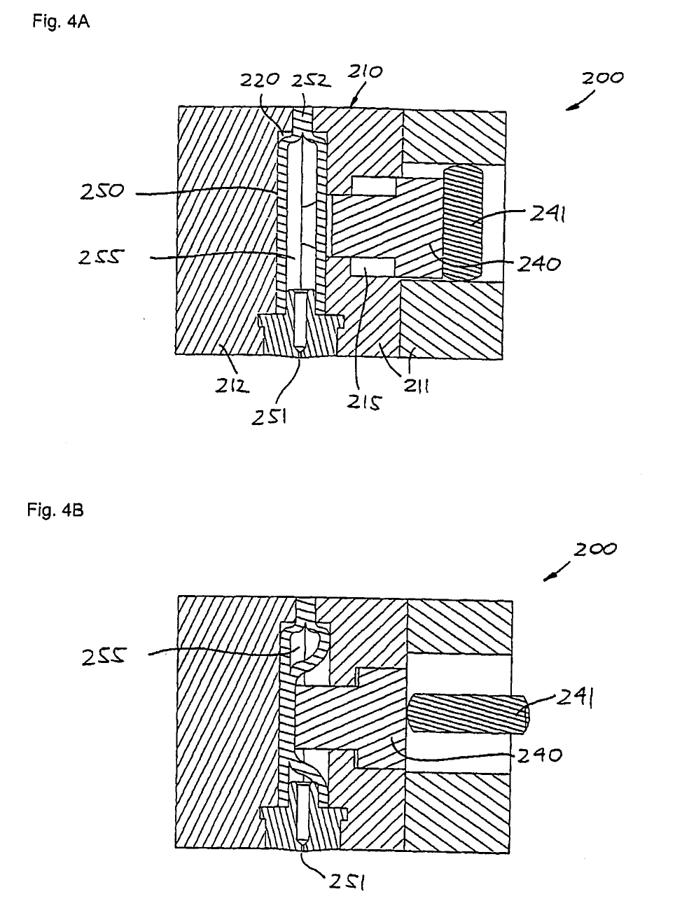

The mounting unit comprises a housing 71 having an open distal end portion 72 and a partially closed proximal end portion 73 in combination forming a mounting (or receiving) cavity 75 for the impulse chamber unit 3, the mounting cavity having a configuration substantially corresponding to the outer configuration of the impulse chamber unit (e.g. circular) to thereby receive the latter in a snug fit (for illustrative purposes a small gap is shown between the two structures). The proximal end portion comprises a proximally extending conduit member 76 in fluid communication with the mounting cavity. The conduit may be in the form of a pointed needle member adapted to penetrate a septum member of a reservoir. The needle may be formed integrally with the housing as shown (e.g. using a suitable polymer) or it may be attached to the housing as a separate component (e.g. a steel needle attached to a polymer housing). The housing further comprises a side opening 74 allowing an impulse generating means to engage a portion of the deformable tube, as well as user gripping means 77 and connection means 78 allowing the mounting unit to be arranged in a therefore adapted impulse generating unit (see below description of FIGS. 4). In the shown embodiment the impulse chamber unit and the mounting unit are releasably connected to each other by corresponding coupling means 63, 79 such as e.g. a threaded connection, a bayonet coupling or a friction coupling provided between the nozzle portion and the distal end of the housing, the nozzle member comprising a circumferential distal flange portion 64 serving as a user gripping means. Further, the distal end surface 52 of the elastomeric tube and the inner surface 80 of the housing end portion are adapted to provide a sealed connection therebetween when the impulse chamber unit is mounted in the mounting unit. This arrangement would allow the mounting unit to be used as a semi disposable connection unit mounted between a reservoir and the impulse chamber unit, into which a fully disposable impulse chamber unit is mounted. For example, a new mounting unit may be used for each new prefilled cartridge used whereas a new impulse chamber unit may be used for each jet injection. However, the two members may alternatively be permanently attached to each other thereby providing a unitary impulse chamber unit. In the shown embodiment the conduit is directly in fluid communication with the impulse chamber, however, in an alternative embodiment the conduit may be axially offset terminating in the sealing area between the tube and the housing thereby allowing the elastomeric tube to serve as a non return valve, i.e. allowing fluid to be forced into the chamber between the tube and the housing end portion, yet preventing fluid from returning during pressure build up in the impulse chamber.

FIG. 3 shows a schematic representation of a two-stage jet injection device. More specifically, the jet injection device 100 comprises a reservoir portion 120, a jet injection portion 140 and a fluid channel 130 there between. The reservoir portion comprises a body portion 126 having a distal end 121 with an outlet 123, and an open proximal end 122 in which a piston 124 is slidably received, the body portion and the piston defining a variable volume reservoir 125 for storing a fluid medical compound, such as insulin. The jet injection unit 140 comprises a housing 141, and a piston 142 movably arranged therein, the housing and the piston defining a variable volume mounting cavity in which an impulse chamber unit 180 of the above-described type is arranged, the impulse chamber unit comprising an impulse chamber 185, an inlet 181 in fluid communication with the reservoir, and a nozzle outlet 182. The device further comprises an impulse generating mechanism (not shown) for moving the piston.

With reference to FIGS. 4A and 4B a mounting device for an impulse chamber unit of the above-discussed types will be described, the mounting device also comprising impulse generating means.

More specifically, the mounting device 200 comprises a housing 210 having first and second portions 211, 212 in combination forming a mounting (or receiving) cavity 220 for an impulse chamber unit 250 with an impulse chamber 255, a nozzle 251 and a needle penetratable inlet portion 252, the mounting cavity comprising a portion substantially corresponding to the outer configuration of the deformable chamber portion to thereby receive the latter in a snug fit. In case an inner mounting unit of the type shown in FIG. 2B is used, the cavity 220 is adapted for receiving the latter, in which case the inner mounting unit can be considered a replaceable portion of a combined mounting device. The two housing portions can be removed from each other (e.g. by means of a hinge) to allow a user to replace the impulse chamber unit, however, advantageously the mounting cavity is in the form of a bore allowing an impulse chamber unit with a circular outer configuration to be easily inserted and replaced. The impulse chamber unit 250 generally corresponds to the embodiment shown in FIG. 2A although the proximal closed end of the elastomeric tube is formed differently.

The housing further comprises a bore or opening 215 in communication with the mounting cavity and wherein a piston member 240 is slidably received. The piston comprises a distal end adapted to engage the elastomeric portion of the impulse chamber unit, and a proximal portion adapted to engage impulse generating means. In the shown embodiment the mounting unit further comprises a cam member 241 pivotably mounted in the housing as well as means for rotating the cam member (not shown). The means for rotating the cam member may be in the form of a releasably strainable torsion spring. When the spring has been actuated (i.e. cocked) with the cam and the piston in an initial position as shown in FIG. 4A, the user may release the spring whereby the cam is rotated 90 degrees to an intermediate position as shown in FIG. 4B thereby moving the piston to a foremost position deforming/compressing the impulse chamber 255 and thereby expelling a jet of fluid from the impulse chamber through the nozzle corresponding to a first-stage jet injection. In the same motion the cam is rotated further 90 degrees whereby the cam and the piston is positioned in an end position substantially corresponding to the initial position as shown in FIG. 4A. The piston may be returned to its initial position either by a spring (not shown) or by means of the elastic forces of the impulse chamber tube. For the next impulse, the cam may be rotated in the same or in the reverse direction. Thereafter fluid drug can be injected from a reservoir as shown in FIG. 3 corresponding to a second-stage injection.

In the embodiment of FIGS. 4A and 4B the impulse chamber tube is fully compressed by the piston 240 thereby essentially blocking flow of drug through the chamber as provided in two-stage jet injection (see below). Correspondingly, it is necessary that the piston is retracted after actuation and that the compressed tube is capable of expanding at least partially after having been compressed. Alternatively, if the tube is not fully compressed it is not necessary to immediately retract the piston just as the tube may be manufactured from a material that is plastically deformed during compression.

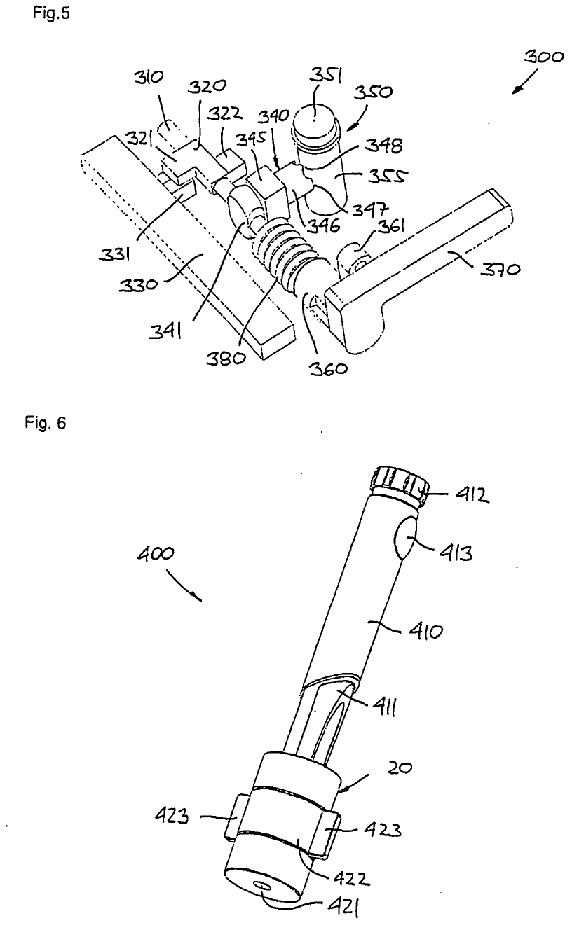

FIG. 5 shows a schematic representation of an impulse chamber unit 350 and an impulse generating mechanism, the impulse chamber unit comprising an elastomeric tube portion 355 and a nozzle 351, the mechanism comprising a cam member and a piston.

More specifically, the mechanism 300 comprises a rotationally mounted axle 310 having a cam member 341 and a stop member 320 fixedly arranged thereon, the stop member comprising first and second arms 321, 322 offset 180 degrees as well as longitudinally relative to each other, a piston 340, a release member 330 with an arm 331 adapted to engage the arms of the stop member, a spring actuation member 360 rotationally arranged on the axle, a spring loaded ratchet stop 361, an actuation member 370 attached to the spring actuation member by a ratchet mechanism allowing the actuation member to uni-directionally rotate the spring actuation member. The different members are arranged in a supporting structure (not shown), e.g. the housing of a mounting unit. The mechanism further comprises a helical torsion spring 380 having a first end attached to the axle and a second end attached to the spring actuation member. The piston comprises a proximal head portion 345 serving as a cam follower, and a distal end 346 adapted to engage an elastomeric tube portion of the impulse chamber unit, the distal end comprising two outer ridge portions 347 and a central ridge portion 348, this configuration serving to deform/compress the elastomeric tube in a controlled manner providing a rapid build-up of pressure in the impulse chamber.

In a situation of use the actuation member 370 is rotated 180 degrees counter clockwise (or less in case a gear mechanism is provided) whereby the spring actuation member is rotated 180 degrees thereby straining (cocking) the spring, which is prevented from rotating the axle due to the release member engaging the stop member of the axle. When the release member is actuated (in the shown embodiment by longitudinal translation towards the spring) the first arm 321 of the stop member is released and the axle is allowed to swiftly rotate 180 degrees until the second arm 322 of the stop member engages the arm of the release member, where after the release member after the next cocking action is ready for the next release by moving the release member in the opposite direction. During rotation of the cam the piston is moved to a foremost position deforming the elastomeric tube portion 355 thereby expelling a jet of fluid from the impulse chamber through the nozzle 351 corresponding to a first-stage jet injection, where after the piston is returned to its initial position, either by spring means (not shown) or the elastic properties of the tube.

With reference to FIG. 6 a two-stage jet injection device 400 is shown. The device comprises a pen-formed proximal portion 410 in which a conventional reservoir cartridge 411 is arranged, and a distal impulse generating portion 420 in which an impulse chamber unit of the type shown in FIG. 1 is mounted, the impulse chamber unit having a proximal inlet in fluid communication with the reservoir and a distal outlet nozzle opening 421. The pen-formed portion may be in the form of a durable device adapted to receive a replaceable cartridge or it may be a prefilled, disposable device. Correspondingly, the proximal portion may have any desirable configuration just as it may be a fully manually operated device or an electronically controlled motorized device. The two portions are releasably connected by convenient means such as e.g. a threaded connection or a bayonet coupling, however, for different configurations of the proximal portion (e.g. when adapted for rear-or side-wards loading of the cartridge) the two portions may be provided as a unitary device. This also applies in case a two-stage jet injection device is provided as a fully disposable device.

The pen portion comprises a rotatable dose setting member 412 for setting a desired dose, e.g. a number of insulin units. The dose setting member cooperates with a dose setting mechanism which simultaneously sets a given dose and stores the energy necessary for a subsequently expelling the set dose of drug from the reservoir, e.g. by straining a spring or compressing a gas. To release the spring, a release knob 413 is provided. For further details in respect of such a mechanism reference is made to applicants EP 1 351 732, hereby incorporated by reference.

The impulse generating portion comprises a rotatable ring member 422 with a pair of wing members 423 allowing the user to rotate the ring e.g. 180 degrees to activate and cock an impulse generator. The latter may be of the type described with reference to FIG. 4C. In a situation of use, the impulse generator is also released by the release knob (e.g. by a rod arranged between the release knob and the impulse generator), this allowing the two stages of the two-stage jet injection to be activated properly with respect to each other as will be described below.

In the following a situation of use will be described. First a cartridge is mounted (or replaced) in the pen portion by disassembling and reassembling the two portions of the device. Alternatively a prefilled pen is mounted. Thereafter a new impulse chamber unit of the type shown in FIG. 1 is removed from its sterile enclosure (not shown) and mounted through a distal opening in the impulse portion, thereby securing it in place. By this action the proximal needle of the impulse chamber unit penetrates the septum of the cartridge thereby establishing fluid communication between the reservoir and the impulse chamber.

The device is now ready for being prepared for injection. First a small dose is set using the dose setting member and subsequently expelled from the reservoir using the release knob. This action corresponds to an “air shot” when a new hypodermic needle is mounted on an injection device, whereby the impulse chamber is filled with drug. The initial small dose should ensure that a small volume of drug oozes from the jet nozzle to indicate that the impulse chamber has been filled. Thereafter the desired dose to be injected is set and the impulse generating means is strained by cocking the ring.

When the dose is selected and the drive means is actuated the user places the nozzle against a skin portion of a subject and actuates the release means 413 whereby a first-stage injection is performed as the impulse chamber is deformed due to release of the impulse generator followed by a second stage-injection due to coupled release of the dose expelling means. Indeed, in case the impulse chamber has been fully compressed the coupling has to ensure proper timing between the two releases providing that compression of the impulse chamber has seized when the second-stage dose injection begins. Depending on the time between the two stages and the elastic properties of the tube, the impulse chamber may have partly regained its initial configuration due to the elastic properties of the tube whereby a small amount of air or fluid may be socked into the impulse chamber through the nozzle, however, it is assumed that this volume will for most practical purposes be neglectable.

In an alternative embodiment (not shown) the dose expelling may be performed manually by the user, typically by depressing an actuation button which may be formed integrally with the above-described dose setting member, for example as disclosed in applicants U.S. pat. No. 6,235,004, hereby incorporated by reference. For such an arrangement, activation of the impulse generating means may be coupled to the initial depression of the actuation button. In a further alternative embodiment (not shown) the impulse generating means may be released (and optionally cocked) automatically as the device is forced against a skin portion of a subject with a given force, this assuring that the outlet nozzle is in proper contact with the skin surface when the first-stage injection is performed. The second-stage injection may subsequently be performed automatically or it may be performed or initiated manually as described above. In a yet further alternative embodiment the impulse generating portion incorporating an impulse chamber is provided as a fully disposable nozzle device which may be used as an equivalent to a traditional subcutaneous needle and in combination with a conventional, non-modified injection device. Such a nozzle device may be provided to the user in a pre-cocked condition or it may be cocked just prior to use, e.g. automatically when the nozzle device is attached to the injection device, when a protective cover is removed from the nozzle device, or when the nozzle device is forced against a skin portion. Release of the impulse generating means may take place automatically as the nozzle device is forced against a skin portion, or manually by the user. The release action may also be coupled to the actuation of a modified injection device as described above.

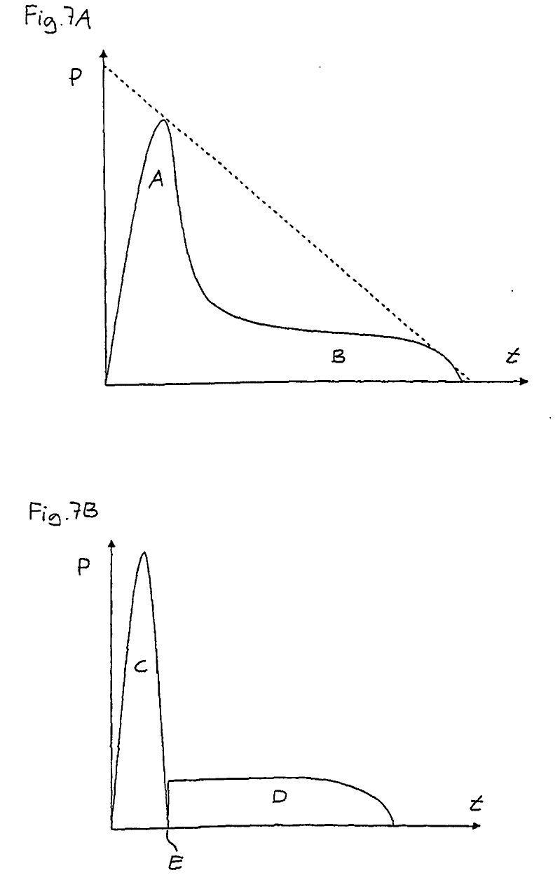

Referring to FIG. 7A, showing the principle pressure-time relationship of a two-stage jet injection of a medical compound, e.g. insulin, using a prior art injection device, the abscissa indicating the time t, and the ordinate axis showing the pressure P. Initially, a jet suitable for penetration of the derma of a patient is expelled under a very high pressure creating an impulse, which is represented by a peak A. The initial impulse subsequently fades out, and the jet-injection continues under a considerably lower pressure as indicated by the portion B. The area under the graph is an indicator of the energy that the expelled fluid delivers to the skin. In FIG. 7A the dashed line symbolizes the pressure time relationship of a typical one-stage jet injection. By comparing the areas under the graphs for the one stage and two-stage jet injections it is obvious that the two stage jet injection delivers considerably less energy at the injection site, and thus is more gentle to the patient. In FIG. 7B the principle pressure-time relationship of a two-stage jet injection corresponding to an aspect of the present invention is shown. The penetration jet is represented by the hump-shaped curve C, and the subsequent injection of the major part of the dose of the medical compound is represented by the flat curve D. There is a distinct break between these two stages of the jet injection, represented by E. The break is caused by the jet impulse chamber being closed of when fully compressed by the piston.

In the above description of the preferred embodiments, the different structures and means providing the described functionality for the different components have been described to a degree to which the concept of the present invention will be apparent to the skilled reader. The detailed mechanical design and specification for the different components are considered the object of a normal design procedure performed by the skilled person along the lines set out in the present specification.

1. An impulse chamber unit (1, 2, 3) comprising:
a deformable chamber portion (10, 50) having an outer surface, the outer surface being substantially free,
a variable-volume impulse chamber (11, 55) defined at least partially by the deformable chamber portion and adapted for containing a volume of a flowable drug,
an outlet nozzle (23, 61) in fluid communication with the impulse chamber and being adapted to be arranged against a skin surface of a subject, and
an inlet (33, 39, 51) in fluid communication with the impulse chamber,
wherein deformation of the chamber portion (10, 50) reduces the volume of the impulse chamber.

2. An impulse chamber unit as in claim 1, wherein the chamber portion is in the form of a deformable polymeric tube comprising proximal and distal end portions.

3. An impulse chamber unit as in claim 2, wherein the outlet nozzle is formed integrally with the polymeric tube.

4. An impulse chamber unit as in claim 2, wherein the tube is formed from a substantially incompressible elastomeric material.

5. An impulse chamber unit as in claim 2, comprising a first closure member (20, 60) attached to the distal end portion, the first closure member comprising the outlet nozzle.

6. An impulse chamber unit as in claim 2, wherein the fluid inlet is provided by a proximal end opening (51) in the polymeric tube.

7. An impulse chamber unit as in claim 2, comprising a first closure member (52) attached to the distal end portion, the first closure member comprising the outlet nozzle, and a second closure member (70) attached relative to the proximal end portion, the second closure member comprising the fluid inlet.

8. An impulse chamber unit as in claim 2, wherein the variable-volume impulse chamber is defined substantially by the deformable chamber portion.

9. An impulse chamber unit as in claim 2, wherein the fluid inlet is in the form of a conduit member (33, 76) projecting from the impulse chamber unit, the conduit member optionally being in the form of a pointed hollow needle.

10. A combination of an impulse chamber unit as in any of claim 1, and a mounting device (60, 200, 420) adapted to replaceably receive the impulse chamber unit in locking engagement.

11. A combination of an impulse chamber unit as in claim 1, and a mounting device (60) adapted to replaceably receive the impulse chamber unit in locking engagement, the mounting device comprising a conduit member projecting from the mounting device and adapted to be arranged in fluid communication with the impulse chamber, the conduit member optionally being in the form of a pointed hollow needle.

12. A combination as in claim 10, wherein the mounting device comprises a mounting cavity adapted to receive the deformable chamber portion in a substantially form-fitting relationship.

13. A combination as in claim 12, wherein the deformable chamber portion has a tubular configuration with a substantially constant outer diameter along the length thereof, the mounting cavity being in the form of a bore having a diameter substantially the same as the deformable chamber portion, the bore comprising an opening (74) allowing an impulse generating element to engage a portion of the deformable chamber portion.

14. A combination as in claim 10, wherein the impulse chamber unit and the mounting device comprise releasable mating coupling means (25, 63, 79) allowing the impulse chamber unit to be secured to the mounting device in a situation of use.

15. A combination as in claim 10, the mounting device further comprising:
a coupling for arranging the fluid inlet in fluid communication with an interior of a drug reservoir, and
an impulse generator (240, 300, 422) for displacing a portion of the deformable chamber portion, thereby reducing the volume of the impulse chamber and thereby expelling an amount of a liquid drug contained in the impulse chamber through the outlet nozzle (251, 421), the impulse generator being adapted to create a pressure within the impulse chamber for injecting the liquid drug through the outlet nozzle and through a skin surface of the subject.

16. A combination (400) as in claim 15, further comprising:
a reservoir (411) comprising a liquid drug,
drive means for expelling an amount of drug from the reservoir at a reduced pressure relative to the impulse generating means,
means (412) for selectable setting a dose of drug to be expelled,
means (422) for actuating the impulse generating means and the drive means, and
actuatable release means (413), whereby actuation of the release means first causes release of the impulse generator thereby expelling an amount of drug at a high pressure from the impulse chamber through the outlet nozzle (421), followed by release of the drive means for subsequent expelling of the set dose from the reservoir via the impulse chamber through the outlet nozzle.

17. A combination (400) as in claim 15, further comprising:
a reservoir (411) comprising a liquid drug,
drive means for expelling an amount of drug from the reservoir at a reduced pressure relative to the impulse generating means,
means (412) for selectable setting a dose of drug to be expelled,
means (422) for actuating the impulse generating means and the drive means, and
actuatable first release means, whereby actuation of the first release means causes release of the impulse generator thereby expelling an amount of drug at a high pressure from the impulse chamber through the outlet nozzle (421), and
actuatable second release means (413), whereby actuation of the second release means causes release of the drive means for subsequent expelling of the set dose from the reservoir via the impulse chamber through the outlet nozzle.

18. A combination as in claim 15, wherein the impulse generator comprises a piston (240, 340) adapted for deforming the deformable chamber portion (250, 350), a rotatable cam (241, 341) for moving the piston, and a drive mechanism for driving the cam.

19. A combination as in claim 18, wherein the drive mechanism comprises a torsion spring (380), an actuator (360, 370) for bringing the torsion spring in an activated state, and a release (330) for releasing the activated spring and thereby rotate the cam.
